(19) United States
(12) Patent Application Publication
(10) Pub. No.: US 2025/0345541 A1
(43) Pub. Date: Nov. 13, 2025

(54) SYSTEMS AND METHODS FOR AUDIO TRANSMISSION OF ENCODED COMPLIANCE INFORMATION

(71) Applicant: ResMed Pty Ltd, Bella Vista (AU)

(72) Inventor: David Andrew Richards, Sydney (AU)

(21) Appl. No.: 19/205,877

(22) Filed: May 12, 2025

Related U.S. Application Data
(60) Provisional application No. 63/646,604, filed on May 13, 2024.

(51) Int. Cl. A61M 16/00; G10L 19/00; G16H 15/00; G16H 40/60

(52) U.S. Cl. CPC A61M 16/024; G10L 19/00; G16H 40/60; G16H 15/00

(57) ABSTRACT

A respiratory therapy device configured to supply pressurized air to an individual during one or more sleep sessions includes an audio transducer; a memory storing machine-readable instructions; and a control system including one or more processors configured to execute the machine-readable instructions to implement a method that includes generating data associated with use of the respiratory therapy device by the individual during the one or more sleep sessions; storing the generated data in the memory; encoding at least a portion of the generated data into audio data that is reproducible as an audible signal; and operating the audio transducer to generate the audible signal.

### CROSS-REFERENCE TO REPLATED APPLICATIONS

This application claims the benefit of, and priority to, U.S. Provisional Patent Application No. 63/646,604 filed on May 13, 2024, which is hereby incorporated by reference herein in its entirety.

### TECHNICAL FIELD

The present disclosure relates generally to systems and methods for operating a respiratory therapy system, and more particularly, to systems and methods for transmitting compliance information from the respiratory therapy system using encoded audio data.

### BACKGROUND

Many individuals suffer from sleep-related and/or respiratory disorders such as, for example, Sleep-Disordered Breathing (SDB), which can include Obstructive Sleep Apnea (OSA), Central Sleep Apnea (CSA), other types of apneas such as mixed apneas and hypopneas, and Respiratory Effort Related Arousal (RERA). These individuals may also suffer from other health conditions (which may be referred to as comorbidities), such as insomnia (characterized by, for example, difficult in initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders. These individuals are often treated using a respiratory therapy system (e.g., a continuous positive airway pressure (CPAP) system), which delivers pressurized air to aid in preventing the individual's airway from narrowing or collapsing during sleep. The respiratory therapy system can include a conduit that delivers the pressurized air from a respiratory therapy device having a flow generator (e.g., a motor), to a user interface coupled to the individual's face. In some cases, data about the individual's use of the respiratory therapy system, and more specifically about the individual's compliance with a therapy plan, is needed by a third party, such as a healthcare provider, a technician, a payor, etc. However, it can be cumbersome for some individuals to connect their respiratory therapy device to their own personal devices (e.g., a smartphone), in order to transmit this information to the third party. The present disclosure is directed to solving this and other problems.

### SUMMARY

According to some implementations of the present disclosure, a respiratory therapy system configured to supply pressurized air to an individual during one or more sleep session comprises an audio transducer, a memory storing machine-reading instructions, and a control system including one or more processors. The one or more processors are configured to execute the machine-readable instructions to generate data associated with use of the respiratory therapy device by the individual during the one or more sleep sessions. The one or more processors are further configured to execute the machine-readable instructions to store the generated data in the memory. The one or more processors are further configured to execute the machine-readable instructions to encode at least a portion of the generated data into audio data that is reproducible as an audible signal. The one or more processors are further configured to execute the machine-readable instructions to operate the audio transducer to generate the audible signal.

According to some implementations of the present disclosure, a method comprises generating data associated with use of the respiratory therapy device by the individual during the one or more sleep sessions. The method further comprises storing the generated data in a memory of the respiratory therapy device. The method further comprises encoding at least a portion of the generated data into audio data that is reproducible as an audible signal. The method further comprises operating an audio transducer of the respiratory therapy device to generate the audible signal.

According to some implementations of the present disclosure, a method comprises establishing an audio connection between (i) a remote device and (ii) a personal device of the individual or of an aide of the individual. The method further comprises receiving an audible signal that is generated by the respiratory therapy device and transmitted to the remote device from the personal device. The method further comprises decoding the audible signal to produce data associated with use of the respiratory therapy device by the individual during the one or more sleep session.

The above summary is not intended to represent each embodiment or every aspect of the present invention. Additional features and benefits of the present invention are apparent from the detailed description and figures set forth below.

### BRIEF DESCRIPTION OF THE DRAWINGS

FIG. 1 is a functional block diagram of a system for transmitting compliance information to a third party, according to some implementations of the present disclosure;

FIG. 2 is a perspective view of the system of FIG. 1, a user of the system, and a bed partner of the user, according to some implementations of the present disclosure;

FIG. 3 illustrates an exemplary timeline for a sleep session, according to some implementations of the present disclosure;

FIG. 4 illustrates an exemplary hypnogram associated with the sleep session of FIG. 3, according to some implementations of the present disclosure;

FIG. 5 illustrates an example compliance report, according to some implementations of the present disclosure;

FIG. 6A is a perspective view of an audio connection established between a personal device of user and a third party, according to some implementations of the present disclosure;

FIG. 6B is a perspective view of the user moving their personal device toward the respiratory therapy device in response to receiving an instructions, according to some implementations of the present disclosure;

FIG. 6C is a perspective view of an audible signal being generated by the respiratory therapy device and transmitted to the third party via the audio connection, according to some implementations of the present disclosure; and

FIG. 7 is a process flow diagram of a method for transmitting compliance information, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

The present disclosure is described with reference to the attached figures, where like reference numerals are used throughout the figures to designate similar or equivalent elements. The figures are not drawn to scale, and are provided merely to illustrate the instant disclosure. Several aspects of the disclosure are described below with reference to example applications for illustration.

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB), Obstructive Sleep Apnea (OSA), Central Sleep Apnea (CSA), other types of apneas, Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders.

Many individuals suffer from sleep-related and/or respiratory disorders, such as Periodic Limb Movement Disorder (PLMD), Restless Leg Syndrome (RLS), Sleep-Disordered Breathing (SDB) such as Obstructive Sleep Apnea (OSA), Central Sleep Apnea (CSA) and other types of apneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), 500Neuromuscular Disease (NMD), and chest wall disorders. Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate, and posterior oropharyngeal wall. Central Sleep Apnea (CSA) is another form of sleep disordered breathing. CSA results when the brain temporarily stops sending signals to the muscles that control breathing. Other types of apneas include hypopnca, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration. A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnca. These events must fulfil both of the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is a further form of SDB. CSR is a disorder of a patient's respiratory controller in which there are rhythmic alternating periods of waxing and waning ventilation known as CSR cycles. CSR is characterized by repetitive de-oxygenation and re-oxygenation of the arterial blood. OHS is defined as the combination of severe obesity and awake chronic hypercapnia, in the absence of other known causes for hypoventilation. Symptoms include dyspnea, morning headache and excessive daytime sleepiness. COPD encompasses any of a group of lower airway diseases that have certain characteristics in common, such as increased resistance to air movement, extended expiratory phase of respiration, and loss of the normal elasticity of the lung. NMD encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

Many of these disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that can occur when the individual is sleeping. A wide variety of types of data can be used to monitor the health of individuals having any of the above types of sleep-related and/or respiratory disorders (or other disorders).

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

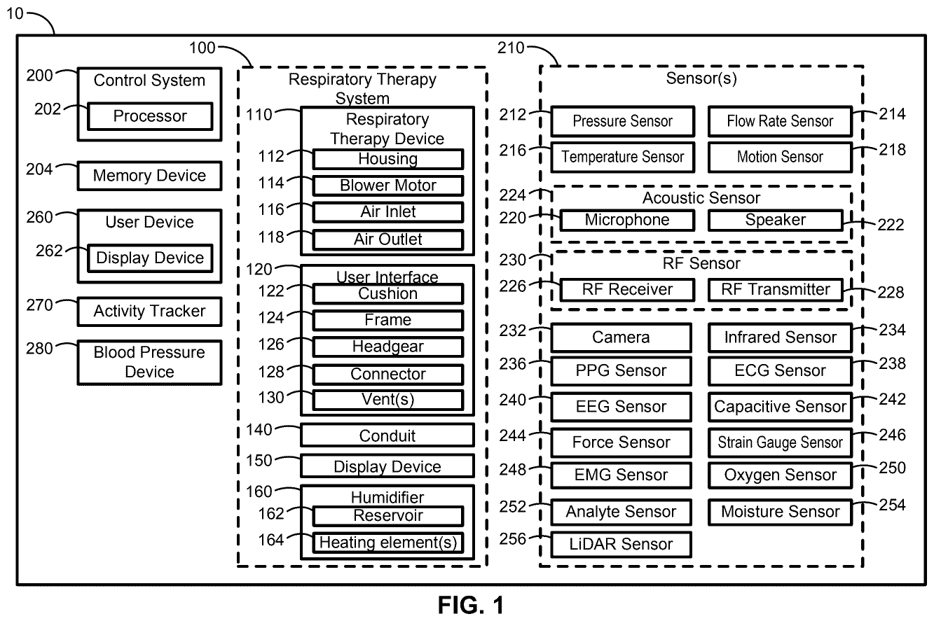

Referring to FIG. 1, a system 10, according to some implementations of the present disclosure, is illustrated. The system 10 can include a respiratory therapy system 100, a control system 200, and a memory device 204. The system 10 may additionally or alternatively include one or more sensors 210, a user device 260, an activity tracker 270, and a blood pressure device 280.

The respiratory therapy system 100 includes a respiratory pressure therapy (RPT) device 110 (referred to herein as respiratory therapy device 110), a user interface 120 (also referred to as a mask or a patient interface), a conduit 140 (also referred to as a tube or an air circuit), a display device 150, and a humidifier 160. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 100 is generally used to treat individuals suffering from one or more sleep-related respiratory disorders (e.g., obstructive sleep apnea, central sleep apnea, or mixed sleep apnea).

The respiratory therapy system 100 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

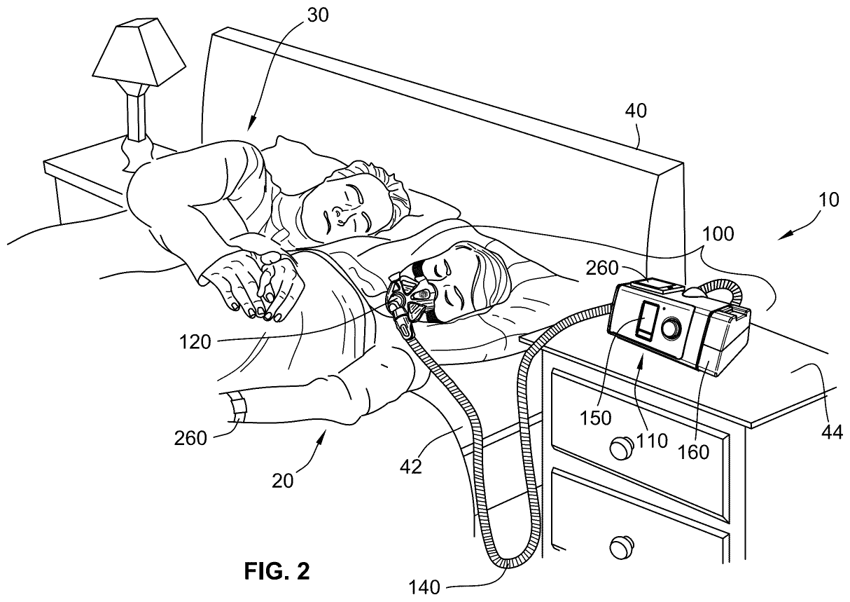

As shown in FIG. 2, the respiratory therapy system 100 can be used to treat a user 20. In this example, the user 20 of the respiratory therapy system 100 and a bed partner 30 are in a bed 40 and are laying on a mattress 42. The user interface 120 can be worn by the user 20 during a sleep session. The respiratory therapy system 100 generally aids in increasing the air pressure in the throat of the user 20 to aid in preventing the airway from closing and/or narrowing during sleep. The respiratory therapy device 110 can be positioned on a nightstand 44 that is directly adjacent to the bed 40 as shown in FIG. 2, or more generally, on any surface or structure that is generally adjacent to the bed 40 and/or the user 20.

Referring back to FIG. 1, the respiratory therapy device 110 is generally used to generate pressurized air that is delivered to a user (e.g., using one or more motors that drive one or more compressors). In some implementations, the respiratory therapy device 110 generates continuous constant air pressure that is delivered to the user. In other implementations, the respiratory therapy device 110 generates two or more predetermined pressures (e.g., a first predetermined air pressure and a second predetermined air pressure). In still other implementations, the respiratory therapy device 110 generates a variety of different air pressures within a predetermined range. For example, the respiratory therapy device 110 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory therapy device 110 can also deliver pressurized air at a predetermined flow rate between, for example, about-20 L/min and about 150 L/min, while maintaining a positive pressure (relative to the ambient pressure).

The respiratory therapy device 110 includes a housing 112, a blower motor 114, an air inlet 116, and an air outlet 118. The blower motor 114 is at least partially disposed or integrated within the housing 112. The blower motor 114 draws air from outside the housing 112 (e.g., atmosphere) via the air inlet 116 and causes pressurized air to flow through the humidifier 160, and through the air outlet 118. In some implementations, the air inlet 116 and/or the air outlet 118 include a cover that is moveable between a closed position and an open position (e.g., to prevent or inhibit air from flowing through the air inlet 116 or the air outlet 118). The housing 112 can also include a vent to allow air to pass through the housing 112 to the air inlet 116. As described below, the conduit 140 is coupled to the air outlet 118 of the respiratory therapy device 110.

The user interface 120 engages a portion of the user's face and delivers pressurized air from the respiratory therapy device 110 to the user's airway to aid in preventing the airway from narrowing and/or collapsing during sleep. This may also increase the user's oxygen intake during sleep. Generally, the user interface 120 engages the user's face such that the pressurized air is delivered to the user's airway via the user's mouth, the user's nose, or both the user's mouth and nose. Together, the respiratory therapy device 110, the user interface 120, and the conduit 140 form an air pathway fluidly coupled with an airway of the user. The pressurized air also increases the user's oxygen intake during sleep. Depending upon the therapy to be applied, the user interface 120 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cmH₂O.

The user interface 120 can include, for example, a cushion 122, a frame 124, a headgear 126, connector 128, and one or more vents 130. The cushion 122 and the frame 124 define a volume of space around the mouth and/or nose of the user. When the respiratory therapy system 100 is in use, this volume space receives pressurized air (e.g., from the respiratory therapy device 110 via the conduit 140) for passage into the airway(s) of the user. The headgear 126 is generally used to aid in positioning and/or stabilizing the user interface 120 on a portion of the user (e.g., the face), and along with the cushion 122 (which, for example, can comprise silicone, plastic, foam, etc.) aids in providing a substantially air-tight seal between the user interface 120 and the user 20. In some implementations the headgear 126 includes one or more straps (e.g., including hook and loop fasteners). The connector 128 is generally used to couple (e.g., connect and fluidly couple) the conduit 140 to the cushion 122 and/or frame 124. Alternatively, the conduit 140 can be directly coupled to the cushion 122 and/or frame 124 without the connector 128. The one or more vents 130 can be used for permitting the escape of carbon dioxide and other gases exhaled by the user 20. The user interface 120 generally can include any suitable number of vents (e.g., one, two, five, ten, etc.).

As shown in FIG. 2, in some implementations, the user interface 120 is a facial mask (e.g., a full-face mask) that covers at least a portion of the nose and mouth of the user 20. Alternatively, the user interface 120 can be a nasal mask that provides air to the nose of the user or a nasal pillow mask that delivers air directly to the nostrils of the user 20. In other implementations, the user interface 120 includes a mouthpiece (e.g., a night guard mouthpiece molded to conform to the teeth of the user, a mandibular repositioning device, etc.).

Referring back to FIG. 1, the conduit 140 (also referred to as an air circuit or tube) allows the flow of air between components of the respiratory therapy system 100, such as between the respiratory therapy device 110 and the user interface 120. In some implementations, there can be separate limbs of the conduit for inhalation and exhalation. In other implementations, a single limb conduit is used for both inhalation and exhalation.

The conduit 140 includes a first end that is coupled to the air outlet 118 of the respiratory therapy device 110. The first end can be coupled to the air outlet 118 of the respiratory therapy device 110 using a variety of techniques (e.g., a press fit connection, a snap fit connection, a threaded connection, etc.). In some implementations, the conduit 140 includes one or more heating elements that heat the pressurized air flowing through the conduit 140 (e.g., heat the air to a predetermined temperature or within a range of predetermined temperatures). Such heating elements can be coupled to and/or imbedded in the conduit 140. In such implementations, the first end can include an electrical contact that is electrically coupled to the respiratory therapy device 110 to power the one or more heating elements of the conduit 140. For example, the electrical contact can be electrically coupled to an electrical contact of the air outlet 118 of the respiratory therapy device 110. In this example, electrical contact of the conduit 140 can be a male connector and the electrical contact of the air outlet 118 can be female connector, or, alternatively, the opposite configuration can be used.

The display device 150 is generally used to display image(s) including still images, video images, or both and/or information regarding the respiratory therapy device 110. For example, the display device 150 can provide information regarding the status of the respiratory therapy device 110 (e.g., whether the respiratory therapy device 110 is on/off, the pressure of the air being delivered by the respiratory therapy device 110, the temperature of the air being delivered by the respiratory therapy device 110, etc.) and/or other information (e.g., a sleep score and/or a therapy score, also referred to as a myAir™ score, such as described in WO 2016/061629 and U.S. Patent Pub. No. 2017/0311879, which are hereby incorporated by reference herein in their entireties, the current date/time, personal information for the user 20, etc.). In some implementations, the display device 150 acts as a human-machine interface (HMI) that includes a graphic user interface (GUI) configured to display the image(s) as an input interface. The display device 150 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 110.

The humidifier 160 is coupled to or integrated in the respiratory therapy device 110 and includes a reservoir 162 for storing water that can be used to humidify the pressurized air delivered from the respiratory therapy device 110. The humidifier 160 includes a one or more heating elements 164 to heat the water in the reservoir to generate water vapor. The humidifier 160 can be fluidly coupled to a water vapor inlet of the air pathway between the blower motor 114 and the air outlet 118, or can be formed in-line with the air pathway between the blower motor 114 and the air outlet 118. For example, air flows from the air inlet 116 through the blower motor 114, and then through the humidifier 160 before exiting the respiratory therapy device 110 via the air outlet 118.

While the respiratory therapy system 100 has been described herein as including each of the respiratory therapy device 110, the user interface 120, the conduit 140, the display device 150, and the humidifier 160, more or fewer components can be included in a respiratory therapy system according to implementations of the present disclosure. For example, a first alternative respiratory therapy system includes the respiratory therapy device 110, the user interface 120, and the conduit 140. As another example, a second alternative system includes the respiratory therapy device 110, the user interface 120, and the conduit 140, and the display device 150. Thus, various respiratory therapy systems can be formed using any portion or portions of the components shown and described herein and/or in combination with one or more other components.

The control system 200 includes one or more processors 202 (hereinafter, processor 202). The control system 200 is generally used to control (e.g., actuate) the various components of the system 10 and/or analyze data obtained and/or generated by the components of the system 10. The processor 202 can be a general or special purpose processor or microprocessor. While one processor 202 is illustrated in FIG. 1, the control system 200 can include any number of processors (e.g., one processor, two processors, five processors, ten processors, etc.) that can be in a single housing, or located remotely from each other. The control system 200 (or any other control system) or a portion of the control system 200 such as the processor 202 (or any other processor(s) or portion(s) of any other control system), can be used to carry out one or more steps of any of the methods described and/or claimed herein. The control system 200 can be coupled to and/or positioned within, for example, a housing of the user device 260, a portion (e.g., the respiratory therapy device 110) of the respiratory therapy system 100, and/or within a housing of one or more of the sensors 210. The control system 200 can be centralized (within one such housing) or decentralized (within two or more of such housings, which are physically distinct). In such implementations including two or more housings containing the control system 200, the housings can be located proximately and/or remotely from each other. The control system 200 (or one or more portions thereof) can be located in the respiratory therapy device 110, in the user device 260 (e.g., as part of a smartphone application), in the cloud (e.g., in a remote device or system connected various components of the system 10 such as the user device 260 and/or the respiratory therapy device 110), and/or in other locations.

The memory device 204 stores machine-readable instructions that are executable by the processor 202 of the control system 200. The memory device 204 can be any suitable computer readable storage device or media, such as, for example, a random or serial access memory device, a hard drive, a solid-state drive, a flash memory device, etc. While one memory device 204 is shown in FIG. 1, the system 10 can include any suitable number of memory devices 204 (e.g., one memory device, two memory devices, five memory devices, ten memory devices, etc.). The memory device 204 can be coupled to and/or positioned within a housing of a respiratory therapy device 110 of the respiratory therapy system 100, within a housing of the user device 260, within a housing of one or more of the sensors 210, or any combination thereof. Like the control system 200, the memory device 204 can be centralized (within one such housing) or decentralized (within two or more of such housings, which are physically distinct).

In some implementations, the memory device 204 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

As described herein, the processor 202 and/or memory device 204 can receive data (e.g., physiological data and/or audio data) from the one or more sensors 210 such that the data for storage in the memory device 204 and/or for analysis by the processor 202. The processor 202 and/or memory device 204 can communicate with the one or more sensors 210 using a wired connection or a wireless connection (e.g., using an RF communication protocol, a Wi-Fi communication protocol, a Bluetooth communication protocol, over a cellular network, etc.). In some implementations, the system 10 can include an antenna, a receiver (e.g., an RF receiver), a transmitter (e.g., an RF transmitter), a transceiver, or any combination thereof. Such components can be coupled to or integrated a housing of the control system 200 (e.g., in the same housing as the processor 202 and/or memory device 204), or the user device 260.

The one or more sensors 210 include a pressure sensor 212, a flow rate sensor 214, temperature sensor 216, a motion sensor 218, a microphone 220, a speaker 222, a radio-frequency (RF) receiver 226, a RF transmitter 228, a camera 232, an infrared (IR) sensor 234, a photoplethysmogram (PPG) sensor 236, an electrocardiogram (ECG) sensor 238, an electroencephalography (EEG) sensor 240, a capacitive sensor 242, a force sensor 244, a strain gauge sensor 246, an electromyography (EMG) sensor 248, an oxygen sensor 250, an analyte sensor 252, a moisture sensor 254, a Light Detection and Ranging (LiDAR) sensor 256, or any combination thereof. Generally, each of the one or more sensors 210 are configured to output sensor data that is received and stored in the memory device 204 or one or more other memory devices.

While the one or more sensors 210 are shown and described as including each of the pressure sensor 212, the flow rate sensor 214, the temperature sensor 216, the motion sensor 218, the microphone 220, the speaker 222, the RF receiver 226, the RF transmitter 228, the camera 232, the IR sensor 234, the PPG sensor 236, the ECG sensor 238, the EEG sensor 240, the capacitive sensor 242, the force sensor 244, the strain gauge sensor 246, the EMG sensor 248, the oxygen sensor 250, the analyte sensor 252, the moisture sensor 254, and the LiDAR sensor 256, more generally, the one or more sensors 210 can include any combination and any number of each of the sensors described and/or shown herein.

As described herein, the system 10 generally can be used to generate physiological data associated with a user (e.g., a user of the respiratory therapy system 100) during a sleep session. The physiological data can be analyzed to generate one or more sleep-related parameters, which can include any parameter, measurement, etc. related to the user during the sleep session. The one or more sleep-related parameters that can be determined for the user 20 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 110, a heart rate, a heart rate variability, movement of the user 20, temperature, EEG activity, EMG activity, arousal, snoring, choking, coughing, whistling, wheezing, or any combination thereof.

The one or more sensors 210 can be used to generate, for example, physiological data, audio data, or both. Physiological data generated by one or more of the sensors 210 can be used by the control system 200 to determine a sleep-wake signal associated with the user 20 during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 210, are described in, for example, WO 2014/047310, U.S. Patent Pub. No. 2014/0088373, WO 2017/132726, WO 2019/122413, WO 2019/122414, U.S. Patent Pub. No. 2020/0383580, and WO 2022/249013, each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 210 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 110, or any combination thereof during the sleep session. The event(s) can include snoring, apneas, central apneas, obstructive apneas, mixed apneas, hypopneas, a mask leak (e.g., from the user interface 120), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 210 can also be used to determine a respiration signal associated with a user during a sleep session. The respiration signal is generally indicative of respiration or breathing of the user during the sleep session. The respiration signal can be indicative of and/or analyzed to determine (e.g., using the control system 200) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleep stage, an apnea-hypopnea index (AHI), pressure settings of the respiratory therapy device 110, or any combination thereof. The one or more events can include snoring, apneas, central apneas, obstructive apneas, mixed apneas, hypopneas, a mask leak (e.g., from the user interface 120), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 210, or from other types of data.

The pressure sensor 212 outputs pressure data that can be stored in the memory device 204 and/or analyzed by the processor 202 of the control system 200. In some implementations, the pressure sensor 212 is an air pressure sensor (e.g., barometric pressure sensor) that generates sensor data indicative of the respiration (e.g., inhaling and/or exhaling) of the user of the respiratory therapy system 100 and/or ambient pressure. In such implementations, the pressure sensor 212 can be coupled to or integrated in the respiratory therapy device 110. The pressure sensor 212 can be, for example, a capacitive sensor, an electromagnetic sensor, a piezoelectric sensor, a strain-gauge sensor, an optical sensor, a potentiometric sensor, or any combination thereof.

The flow rate sensor 214 outputs flow rate data that can be stored in the memory device 204 and/or analyzed by the processor 202 of the control system 200. Examples of flow rate sensors (such as, for example, the flow rate sensor 214) are described in International Publication No. WO 2012/012835 and U.S. Pat. No. 10,328,219, both of which are hereby incorporated by reference herein in their entireties. In some implementations, the flow rate sensor 214 is used to determine an air flow rate from the respiratory therapy device 110, an air flow rate through the conduit 140, an air flow rate through the user interface 120, or any combination thereof. In such implementations, the flow rate sensor 214 can be coupled to or integrated in the respiratory therapy device 110, the user interface 120, or the conduit 140. The flow rate sensor 214 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 214 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In some examples, the pressure sensor 212 can be used to determine a blood pressure of a user.

The temperature sensor 216 outputs temperature data that can be stored in the memory device 204 and/or analyzed by the processor 202 of the control system 200. In some implementations, the temperature sensor 216 generates temperatures data indicative of a core body temperature of the user 20, a skin temperature of the user 20, a temperature of the air flowing from the respiratory therapy device 110 and/or through the conduit 140, a temperature in the user interface 120, an ambient temperature, or any combination thereof. The temperature sensor 216 can be, for example, a thermocouple sensor, a thermistor sensor, a silicon band gap temperature sensor or semiconductor-based sensor, a resistance temperature detector, or any combination thereof.

The motion sensor 218 outputs motion data that can be stored in the memory device 204 and/or analyzed by the processor 202 of the control system 200. The motion sensor 218 can be used to detect movement of the user 20 during the sleep session, and/or detect movement of any of the components of the respiratory therapy system 100, such as the respiratory therapy device 110, the user interface 120, or the conduit 140. The motion sensor 218 can include one or more inertial sensors, such as accelerometers, gyroscopes, and magnetometers. In some implementations, the motion sensor 218 can comprise an acoustic sensor (such as the acoustic sensor 224 discussed herein) and/or an RF sensor (such as the RF sensor 230 discussed herein), which can generate motion data as further discussed herein. In such implementations, the motion sensor 218, the acoustic sensor, and/or the RF sensor can be disposed in a portable device, such as the user device 260. Further, while FIG. 1 and FIG. 2 show the respiratory therapy device 110 as including its own display device 150, in some implementations the respiratory therapy device 110 may not include its own display device, as is discussed herein. In some implementations, the motion sensor 218 alternatively or additionally generates one or more signals representing bodily movement of the user, from which may be obtained a signal representing a sleep state of the user, for example, via a respiratory movement of the user. In some implementations, the motion data from the motion sensor 218 can be used in conjunction with additional data from another one of the sensors 210 to determine the sleep state of the user.

The microphone 220 outputs sound and/or audio data that can be stored in the memory device 204 and/or analyzed by the processor 202 of the control system 200. The audio data generated by the microphone 220 is reproducible as one or more sound(s) during a sleep session (e.g., sounds from the user 20). The audio data form the microphone 220 can also be used to identify (e.g., using the control system 200) an event experienced by the user during the sleep session, as described in further detail herein. The microphone 220 can be coupled to or integrated in the respiratory therapy device 110, the user interface 120, the conduit 140, or the user device 260. The microphone 220 can be coupled to or integrated in a wearable device, such as a smartwatch, smart glasses, earphones or ear buds, or other head wearable device. In some implementations, the system 10 includes a plurality of microphones (e.g., two or more microphones and/or an array of microphones with beamforming) such that sound data generated by each of the plurality of microphones can be used to discriminate the sound data generated by another of the plurality of microphones.

The speaker 222 outputs sound waves that are audible to a user of the system 10 (e.g., the user 20 of FIG. 2). The speaker 222 can be used, for example, as an alarm clock or to play an alert or message to the user 20 (e.g., in response to an event). In some implementations, the speaker 222 can be used to communicate the audio data generated by the microphone 220 to the user. The speaker 222 can be coupled to or integrated in the respiratory therapy device 110, the user interface 120, the conduit 140, or the user device 260, and/or can be coupled to or integrated in a wearable device, such as a smartwatch, smart glasses, earphones or ear buds, or other head wearable device.

The microphone 220 and the speaker 222 can be used as separate devices. In some implementations, the microphone 220 and the speaker 222 can be combined into an acoustic sensor 224 (e.g., a sonar sensor), as described in, for example, WO 2018/050913, WO 2020/104465, U.S. Pat. App. Pub. No. 2022/0007965, each of which is hereby incorporated by reference herein in its entirety. In such implementations, the speaker 222 generates or emits sound waves at a predetermined interval and the microphone 220 detects the reflections of the emitted sound waves from the speaker 222. The sound waves generated or emitted by the speaker 222 have a frequency that is not audible to the human ear (e.g., below 20 Hz or above around 18 kHz) so as not to disturb the sleep of the user 20 or the bed partner 30. Based at least in part on the data from the microphone 220 and/or the speaker 222, the control system 200 can determine a location of the user 20 and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 110, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 210 include (i) a first microphone that is the same as, or similar to, the microphone 220, and is integrated in the acoustic sensor 224 and (ii) a second microphone that is the same as, or similar to, the microphone 220, but is separate and distinct from the first microphone that is integrated in the acoustic sensor 224.

The RF transmitter 228 generates and/or emits radio waves having a predetermined frequency and/or a predetermined amplitude (e.g., within a high frequency band, within a low frequency band, long wave signals, short wave signals, etc.). The RF receiver 226 detects the reflections of the radio waves emitted from the RF transmitter 228, and this data can be analyzed by the control system 200 to determine a location of the user and/or one or more of the sleep-related parameters described herein. An RF receiver (either the RF receiver 226 and the RF transmitter 228 or another RF pair) can also be used for wireless communication between the control system 200, the respiratory therapy device 110, the one or more sensors 210, the user device 260, or any combination thereof. While the RF receiver 226 and RF transmitter 228 are shown as being separate and distinct elements in FIG. 1, in some implementations, the RF receiver 226 and RF transmitter 228 are combined as a part of an RF sensor 230 (e.g., a RADAR sensor). In some such implementations, the RF sensor 230 includes a control circuit. The format of the RF communication can be Wi-Fi, Bluetooth, or the like.

In some implementations, the RF sensor 230 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 230. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 232 outputs image data reproducible as one or more images (e.g., still images, video images, thermal images, or any combination thereof) that can be stored in the memory device 204. The image data from the camera 232 can be used by the control system 200 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 232 can be used to, for example, identify a location of the user, to determine chest movement of the user, to determine air flow of the mouth and/or nose of the user, to determine a time when the user enters the bed, and to determine a time when the user exits the bed. In some implementations, the camera 232 includes a wide-angle lens or a fisheye lens.

The IR sensor 234 outputs infrared image data reproducible as one or more infrared images (e.g., still images, video images, or both) that can be stored in the memory device 204. The infrared data from the IR sensor 234 can be used to determine one or more sleep-related parameters during a sleep session, including a temperature of the user 20 and/or movement of the user 20. The IR sensor 234 can also be used in conjunction with the camera 232 when measuring the presence, location, and/or movement of the user 20. The IR sensor 234 can detect infrared light having a wavelength between about 700 nm and about 1 mm, for example, while the camera 232 can detect visible light having a wavelength between about 380 nm and about 740 nm.

The PPG sensor 236 outputs physiological data associated with the user 20 that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 236 can be worn by the user 20, embedded in clothing and/or fabric that is worn by the user 20, embedded in and/or coupled to the user interface 120 and/or its associated headgear (e.g., straps, etc.), etc.

The ECG sensor 238 outputs physiological data associated with electrical activity of the heart of the user 20. In some implementations, the ECG sensor 238 includes one or more electrodes that are positioned on or around a portion of the user 20 during the sleep session. The physiological data from the ECG sensor 238 can be used, for example, to determine one or more of the sleep-related parameters described herein.

The EEG sensor 240 outputs physiological data associated with electrical activity of the brain of the user 20. In some implementations, the EEG sensor 240 includes one or more electrodes that are positioned on or around the scalp of the user 20 during the sleep session. The physiological data from the EEG sensor 240 can be used, for example, to determine a sleep state and/or a sleep stage of the user 20 at any given time during the sleep session. In some implementations, the EEG sensor 240 can be integrated into the user interface 120, into associated headgear (e.g., straps, etc.), into a head band or other head-worn sensor device, etc.

The capacitive sensor 242, the force sensor 244, and the strain gauge sensor 246 output data that can be stored in the memory device 204 and used/analyzed by the control system 200 to determine, for example, one or more of the sleep-related parameters described herein. The EMG sensor 248 outputs physiological data associated with electrical activity produced by one or more muscles. The oxygen sensor 250 outputs oxygen data indicative of an oxygen concentration of gas (e.g., in the conduit 140 or at the user interface 120). The oxygen sensor 250 can be, for example, an ultrasonic oxygen sensor, an electrical oxygen sensor, a chemical oxygen sensor, an optical oxygen sensor, a pulse oximeter (e.g., SpO₂ sensor), or any combination thereof.

The analyte sensor 252 can be used to detect the presence of an analyte in the exhaled breath of the user 20. The data output by the analyte sensor 252 can be stored in the memory device 204 and used by the control system 200 to determine the identity and concentration of any analytes in the breath of the user. In some implementations, the analyte sensor 252 is positioned near a mouth of the user to detect analytes in breath exhaled from the user's mouth. For example, when the user interface 120 is a facial mask that covers the nose and mouth of the user, the analyte sensor 252 can be positioned within the facial mask to monitor the user's mouth breathing. In other implementations, such as when the user interface 120 is a nasal mask or a nasal pillow mask, the analyte sensor 252 can be positioned near the nose of the user to detect analytes in breath exhaled through the user's nose. In still other implementations, the analyte sensor 252 can be positioned near the user's mouth when the user interface 120 is a nasal mask or a nasal pillow mask. In this implementation, the analyte sensor 252 can be used to detect whether any air is inadvertently leaking from the user's mouth and/or the user interface 120. In some implementations, the analyte sensor 252 is a volatile organic compound (VOC) sensor that can be used to detect carbon-based chemicals or compounds. In some implementations, the analyte sensor 252 can also be used to detect whether the user is breathing through their nose or mouth. For example, if the data output by an analyte sensor 252 positioned near the mouth of the user or within the facial mask (e.g., in implementations where the user interface 120 is a facial mask) detects the presence of an analyte, the control system 200 can use this data as an indication that the user is breathing through their mouth.

The moisture sensor 254 outputs data that can be stored in the memory device 204 and used by the control system 200. The moisture sensor 254 can be used to detect moisture in various areas surrounding the user (e.g., inside the conduit 140 or the user interface 120, near the user's face, near the connection between the conduit 140 and the user interface 120, near the connection between the conduit 140 and the respiratory therapy device 110, etc.). Thus, in some implementations, the moisture sensor 254 can be coupled to or integrated in the user interface 120 or in the conduit 140 to monitor the humidity of the pressurized air from the respiratory therapy device 110. In other implementations, the moisture sensor 254 is placed near any area where moisture levels need to be monitored. The moisture sensor 254 can also be used to monitor the humidity of the ambient environment surrounding the user, for example, the air inside the bedroom.

The LiDAR sensor 256 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 256 can measure and map an area extending 5 meters or more away from the sensor. The LiDAR data can be fused with point cloud data estimated by an electromagnetic RADAR sensor, for example. The LiDAR sensor(s) 256 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 210 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 210 can be integrated in and/or coupled to any one or more of the components of the system 10, including the respiratory therapy device 110, the user interface 120, the conduit 140, the humidifier 160, the control system 200, the user device 260, the activity tracker 270, or any combination thereof. For example, the microphone 220 and the speaker 222 can be integrated in and/or coupled to the user device 260 and the pressure sensor 212 and/or flow rate sensor 214 are integrated in and/or coupled to the respiratory therapy device 110. In some implementations, at least one of the one or more sensors 210 is not coupled to the respiratory therapy device 110, the control system 200, or the user device 260, and is positioned generally adjacent to the user 20 during the sleep session (e.g., positioned on or in contact with a portion of the user 20, worn by the user 20, coupled to or positioned on the nightstand, coupled to the mattress, coupled to the ceiling, etc.).

One or more of the respiratory therapy device 110, the user interface 120, the conduit 140, the display device 150, and the humidifier 160 can contain one or more sensors (e.g., a pressure sensor, a flow rate sensor, or more generally any of the other sensors 210 described herein). These one or more sensors can be used, for example, to measure the air pressure and/or flow rate of pressurized air supplied by the respiratory therapy device 110.

The data from the one or more sensors 210 can be analyzed (e.g., by the control system 200) to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 210, or from other types of data.

The user device 260 includes a display device 262. The user device 260 can be, for example, a mobile device such as a smartphone, a tablet computer, a gaming console, a smartwatch, a laptop computer, or the like. In some implementations, the user device 260 is a portable device, such as a smartphone, a tablet computer, a smartwatch, a laptop computer, etc. Alternatively, the user device 260 can be an external sensing system, a television (e.g., a smart television) or another smart home device (e.g., a smart speaker(s) such as Google Home, Amazon Echo, Alexa etc.). In some implementations, the user device is a wearable device (e.g., a smartwatch). The display device 262 is generally used to display image(s) including still images, video images, or both. In some implementations, the display device 262 acts as a human-machine interface (HMI) that includes a graphic user interface (GUI) configured to display the image(s) and an input interface. The display device 262 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 260. In some implementations, one or more user devices can be used by and/or included in the system 10. As shown in FIG. 2, the user device 260 can include a smartphone that is received in a dock of the respiratory therapy device 110, as is discussed in more detail herein.

In some implementations, the system 10 also includes the activity tracker 270. The activity tracker 270 is generally used to aid in generating physiological data associated with the user. The activity tracker 270 can include one or more of the sensors 210 described herein, such as, for example, the motion sensor 218 (e.g., one or more accelerometers and/or gyroscopes), the PPG sensor 236, and/or the ECG sensor 238. The physiological data from the activity tracker 270 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 270 is coupled (e.g., electronically or physically) to the user device 260.

In some implementations, the activity tracker 270 is a wearable device that can be worn by the user, such as a smartwatch, a wristband, a ring, or a patch. For example, referring to FIG. 2, the activity tracker 270 is worn on a wrist of the user 20. The activity tracker 270 can also be coupled to or integrated a garment or clothing that is worn by the user. Alternatively still, the activity tracker 270 can also be coupled to or integrated in (e.g., within the same housing) the user device 260. More generally, the activity tracker 270 can be communicatively coupled with, or physically integrated in (e.g., within a housing), the control system 200, the memory device 204, the respiratory therapy system 100, and/or the user device 260.

In some implementations, the system 10 also includes the blood pressure device 280. The blood pressure device 280 is generally used to aid in generating cardiovascular data for determining one or more blood pressure measurements associated with the user 20. The blood pressure device 280 can include at least one of the one or more sensors 210 to measure, for example, a systolic blood pressure component and/or a diastolic blood pressure component.

In some implementations, the blood pressure device 280 is a sphygmomanometer including an inflatable cuff that can be worn by the user 20 and a pressure sensor (e.g., the pressure sensor 212 described herein). For example, in the example of FIG. 2, the blood pressure device 280 can be worn on an upper arm of the user 20. In such implementations where the blood pressure device 280 is a sphygmomanometer, the blood pressure device 280 also includes a pump (e.g., a manually operated bulb) for inflating the cuff. In some implementations, the blood pressure device 280 is coupled to the respiratory therapy device 110 of the respiratory therapy system 100, which in turn delivers pressurized air to inflate the cuff. More generally, the blood pressure device 280 can be communicatively coupled with, and/or physically integrated in (e.g., within a housing), the control system 200, the memory device 204, the respiratory therapy system 100, the user device 260, and/or the activity tracker 270.

In other implementations, the blood pressure device 280 is an ambulatory blood pressure monitor communicatively coupled to the respiratory therapy system 100. An ambulatory blood pressure monitor includes a portable recording device attached to a belt or strap worn by the user 20 and an inflatable cuff attached to the portable recording device and worn around an arm of the user 20. The ambulatory blood pressure monitor is configured to measure blood pressure between about every fifteen minutes to about thirty minutes over a 24-hour or a 48-hour period. The ambulatory blood pressure monitor may measure heart rate of the user 20 at the same time. These multiple readings are averaged over the 24-hour period. The ambulatory blood pressure monitor determines any changes in the measured blood pressure and heart rate of the user 20, as well as any distribution and/or trending patterns of the blood pressure and heart rate data during a sleeping period and an awakened period of the user 20. The measured data and statistics may then be communicated to the respiratory therapy system 100.

The blood pressure device 280 maybe positioned external to the respiratory therapy system 100, coupled directly or indirectly to the user interface 120, coupled directly or indirectly to a headgear associated with the user interface 120, or inflatably coupled to or about a portion of the user 20. The blood pressure device 280 is generally used to aid in generating physiological data for determining one or more blood pressure measurements associated with a user, for example, a systolic blood pressure component and/or a diastolic blood pressure component. In some implementations, the blood pressure device 280 is a sphygmomanometer including an inflatable cuff that can be worn by a user and a pressure sensor (e.g., the pressure sensor 212 described herein).

In some implementations, the blood pressure device 280 is an invasive device which can continuously monitor arterial blood pressure of the user 20 and take an arterial blood sample on demand for analyzing gas of the arterial blood. In some other implementations, the blood pressure device 280 is a continuous blood pressure monitor, using a radio frequency sensor and capable of measuring blood pressure of the user 20 once very few seconds (e.g., every 3 seconds, every 5 seconds, every 7 seconds, etc.) The radio frequency sensor may use continuous wave, frequency-modulated continuous wave (FMCW with ramp chirp, triangle, sinewave), other schemes such as PSK, FSK etc., pulsed continuous wave, and/or spread in ultra-wideband ranges (which may include spreading, PRN codes or impulse systems).

While the control system 200 and the memory device 204 are described and shown in FIG. 1 as being a separate and distinct component of the system 10, in some implementations, the control system 200 and/or the memory device 204 are integrated in the user device 260 and/or the respiratory therapy device 110. Thus, the control system 200 and/or the memory device 204 can be disposed within the housing 112 of the respiratory therapy device 110. Alternatively, in some implementations, the control system 200 or a portion thereof (e.g., the processor 202) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 10 is shown as including all of the components described above, more or fewer components can be included in a system according to implementations of the present disclosure. For example, a first alternative system includes the control system 200, the memory device 204, and at least one of the one or more sensors 210 and does not include the respiratory therapy system 100. As another example, a second alternative system includes the control system 200, the memory device 204, at least one of the one or more sensors 210, and the user device 260. As yet another example, a third alternative system includes the control system 200, the memory device 204, the respiratory therapy system 100, at least one of the one or more sensors 210, and the user device 260. Thus, various systems can be formed using any portion or portions of the components shown and described herein and/or in combination with one or more other components.

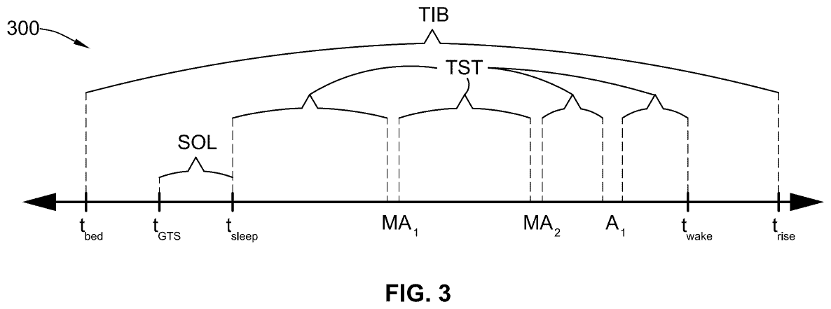

Referring now to FIG. 3, as used herein, a sleep session can be defined multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 262 of the user device 260 (FIG. 1) to manually initiate or terminate the sleep session.

Generally, the sleep session includes any point in time after the user has laid or sat down in the bed (or another area or object on which they intend to sleep) and has turned on the respiratory therapy device 110 and donned the user interface 120. The sleep session can thus include time periods (i) when the user is using the respiratory therapy system 100, but before the user attempts to fall asleep (for example when the user lays in the bed reading a book); (ii) when the user begins trying to fall asleep but is still awake; (iii) when the user is in a light sleep (also referred to as stage 1 and stage 2 of non-rapid eye movement (NREM) sleep); (iv) when the user is in a deep sleep (also referred to as slow-wave sleep, SWS, or stage 3 of NREM sleep); (v) when the user is in rapid eye movement (REM) sleep; (vi) when the user is periodically awake between light sleep, deep sleep, or REM sleep; or (vii) when the user wakes up and does not fall back asleep. The sleep session may also be referred to as a therapy session, or may comprise a therapy session, which can be understood to be the period of time within the sleep session during which the individual is engaged in respiratory therapy (e.g., the use of a respiratory therapy system).

The sleep session is generally defined as ending once the user removes the user interface 120, turns off the respiratory therapy device 110, and gets out of bed. In some implementations, the sleep session can include additional periods of time, or can be limited to only some of the above-disclosed time periods. For example, the sleep session can be defined to encompass a period of time beginning when the respiratory therapy device 110 begins supplying the pressurized air to the airway or the user, ending when the respiratory therapy device 110 stops supplying the pressurized air to the airway of the user, and including some or all of the time points in between, when the user is asleep or awake.

FIG. 3 illustrates an exemplary timeline 300 for a sleep session is illustrated. The timeline 300 includes an enter bed time (t_{bed}), a go-to-sleep time (t_{GTS}), an initial sleep time (tₛₗₑₑₚ), a first micro-awakening MA₁, a second micro-awakening MA₂, an awakening A, a wake-up time (t_{wake}), and a rising time (tᵣᵢₛₑ).

The enter bed time t_{bed} is associated with the time that the user initially enters the bed (e.g., bed 40 in FIG. 2) prior to falling asleep (e.g., when the user lies down or sits in the bed). The enter bed time t_{bed} can be identified based at least in part on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 170, etc.). The initial sleep time (tₛₗₑₑₚ) is the time that the user initially falls asleep. For example, the initial sleep time (t sleep) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based at least in part on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based at least in part on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based at least in part on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based at least in part on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}), or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based at least in part on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 300 of FIG. 3, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

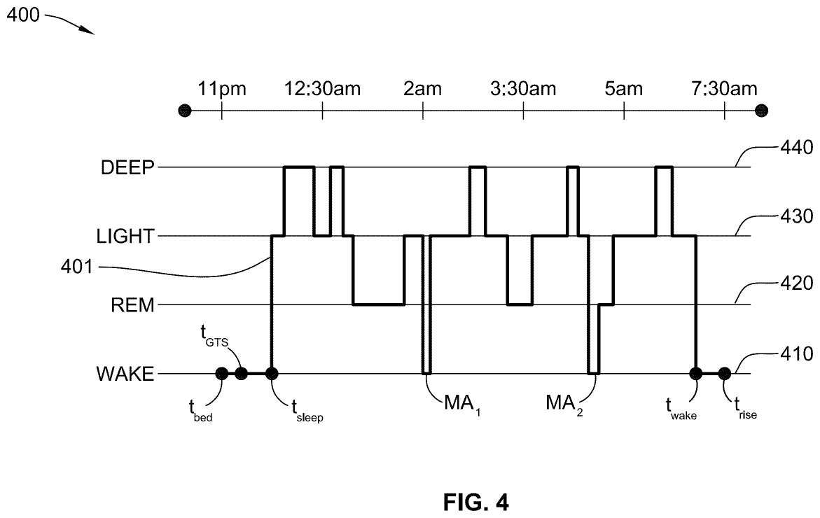

Referring to FIG. 4, an exemplary hypnogram 400 corresponding to the timeline 300 (FIG. 3), according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410-440 is indicative of the sleep stage at any given time during the sleep session.

The sleep-wake signal 401 can be generated based at least in part on physiological data associated with the user (e.g., generated by one or more of the sensors 210 described herein). The sleep-wake signal can be indicative of one or more sleep stages, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration amplitude ratio, an inspiration-expiration duration ratio, a number of events per hour, a pattern of events, or any combination thereof. Information describing the sleep-wake signal can be stored in the memory device 204.

The hypnogram 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening docs not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based at least in part on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 210 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based at least in part on, for example, data generated by the motion sensor 218, the microphone 220, the camera 232, or any combination thereof. The go-to-sleep time can be determined based at least in part on, for example, data from the motion sensor 218 (e.g., data indicative of no movement by the user), data from the camera 232 (e.g., data indicative of no movement by the user and/or that the user has turned off the lights), data from the microphone 220 (e.g., data indicative of the using turning off a TV), data from the user device 170 (e.g., data indicative of the user no longer using the user device 170), data from the pressure sensor 212 and/or the flow rate sensor 214 (e.g., data indicative of the user turning on the respiratory therapy device 110, data indicative of the user donning the user interface 120, etc.), or any combination thereof.

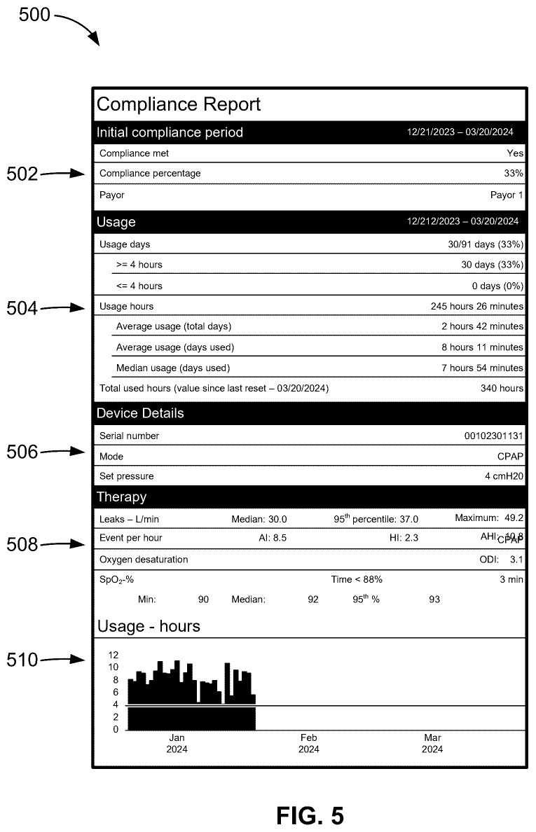

FIG. 5 illustrates a compliance report 500 with details associated with an individual's use of the respiratory therapy system 100. The compliance report 500 may be generated for a variety of different reasons. For example, in some cases health insurance providers and/or other payors may require proof of an individual's compliance with a prescribed use of the respiratory therapy system 100 prior to paying for the purchase or rental of the respiratory therapy system 100. The compliance report 500 (or data and/or information used to produce the compliance report 500) can be transmitted to a remote third party, which could be the payor, a separate entity (such as a healthcare provider or a technician associated with the respiratory therapy system 100) that processes the compliance report 500 (and/or the underlying data/information) and sends the compliance report 500 (and/or the underlying data/information) to the payor, or any other suitable remote entity.

The compliance report 500 shows information associated with the individual's respiratory therapy system, as well as the value of a variety of different metrics. In a first section 502, the compliance report 500 shows the applicable time period (which may be a date range, a specific number of days, a specific number of weeks, etc.), an indication of whether the individual satisfied a predetermined compliance threshold for the period of time, the percentage of days within the time period that the compliance threshold was met for that day, and the identity of the payor.

In a second section 504, the compliance report 500 shows usage information for the time period. The usage information includes the number of days (and/or the percentage of total days within the time period) that the respiratory therapy system 100 was used (which is thus also the number of sleep sessions during the time period when the respiratory therapy system 100 was used); the number of days (and/or the percentage of total days within the time period) that the respiratory therapy system 100 was used for at least a threshold amount of time (which is 4 hours in the illustrated implementation); the number of days (and/or the percentage of the time period) that the respiratory therapy system 100 was not used for at least the threshold amount of time; the total number of hours that the respiratory therapy system 100 was used during the time period; the average duration of use per day over all days within the time period; the average duration of use per day over only days within the time period that the respiratory therapy system 100 was used (which is generally equivalent to the average duration of use per sleep session over all sleep session when the respiratory therapy system 100 was used); the median duration of use per day (and/or sleep session) over only days (and/or sleep sessions) within the time period that the respiratory therapy system 100 was used; and the total number of hours that the respiratory therapy system 100 has been used.

In a third section 506, the compliance report 500 includes information about the respiratory therapy device 110 of the respiratory therapy system. This information includes a unique identifier of the respiratory therapy device 110 (such as a serial number), the mode of operation of the respiratory therapy device 110 (e.g., APAP vs. BPAP vs. CPAP, etc.), and the set pressure of the respiratory therapy device 110 (which can be a prescribed therapy pressure that the respiratory therapy device 110 tries to maintain).

In a fourth section 508, the compliance report 500 includes information about the therapy provided by the respiratory therapy system 100. This information includes information related to air leaking from any part of the respiratory therapy system 100, such as the respiratory therapy device 110, the user interface 120, the conduit 140, etc. The air leak information includes the median rate of the air leak (measured in, for example, liters per minute), the 95^{th} percentile of the rate of the air leak, and the maximum rate of the air leak. The information in the fourth section 508 also includes information about respiratory events experienced by the individual, such as the apnea index (e.g., apnea events per hour), the hypopnea index (e.g., hypopnea events per hour), and the apnea-hypopnea index (e.g., apnea and hypopnea events per hour). The fourth section 508 also includes information about the individual's oxygen levels, including the oxygen desaturation index (e.g., oxygen desaturation events per hour), the minimum oxygen saturation percentage (SpO₂%), the median oxygen saturation percentage, the 95^{th} percentile oxygen saturation percentage, and the total amount of time where the oxygen saturation percentage was less than a threshold amount (88% in the illustrated implementation). Finally, the compliance report 500 includes a plot 510 showing the number of hours of use per day over a period of time. The compliance report 500 can also include any other information.

The information in the compliance report 500 can be based on data that is generated by the respiratory therapy system 100 during use of the respiratory therapy system 100 by the individual. Generally, the respiratory therapy system 100 transmits the generated data to the remote third party, who can then generate the compliance report 500. In other cases, the respiratory therapy system 100 generates the compliance report 500 and transmits the compliance report 500 to the remote third party, in some cases in addition to the data. In either case, the respiratory therapy system 100 must generally be connected to an external network in order to transmit the compliance report 500 and/or the underlying data. This connection can be via a network connection (e.g., a wired connection such as ethernet, a wireless connection such as Wi-Fi, etc.), a cellular connection (e.g., via a cellular chip inside the respiratory therapy device 110), via a personal device of the individual (e.g., a Bluetooth connection between the respiratory therapy device 110 and the individual's smartphone), etc.

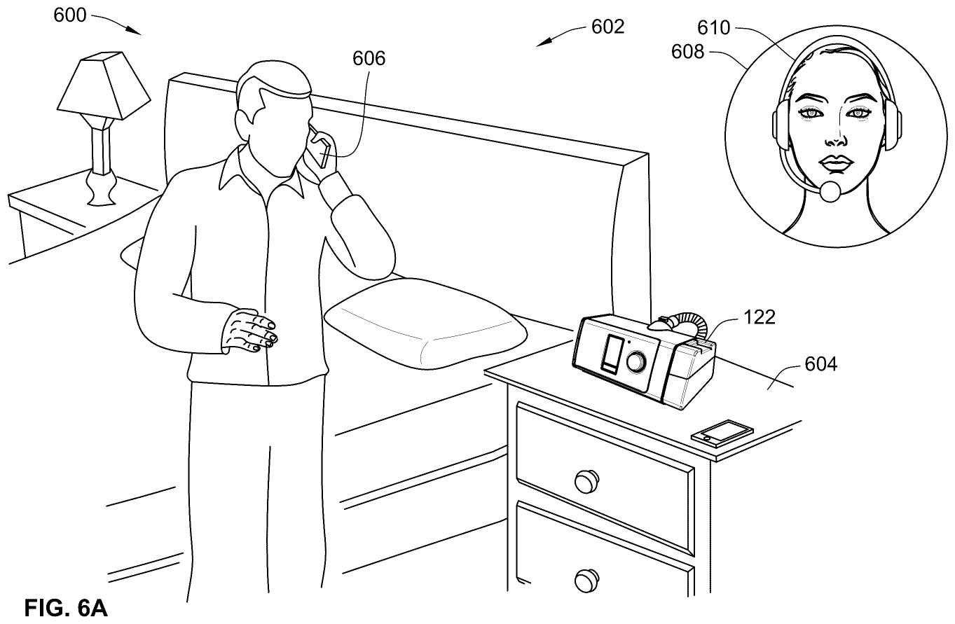

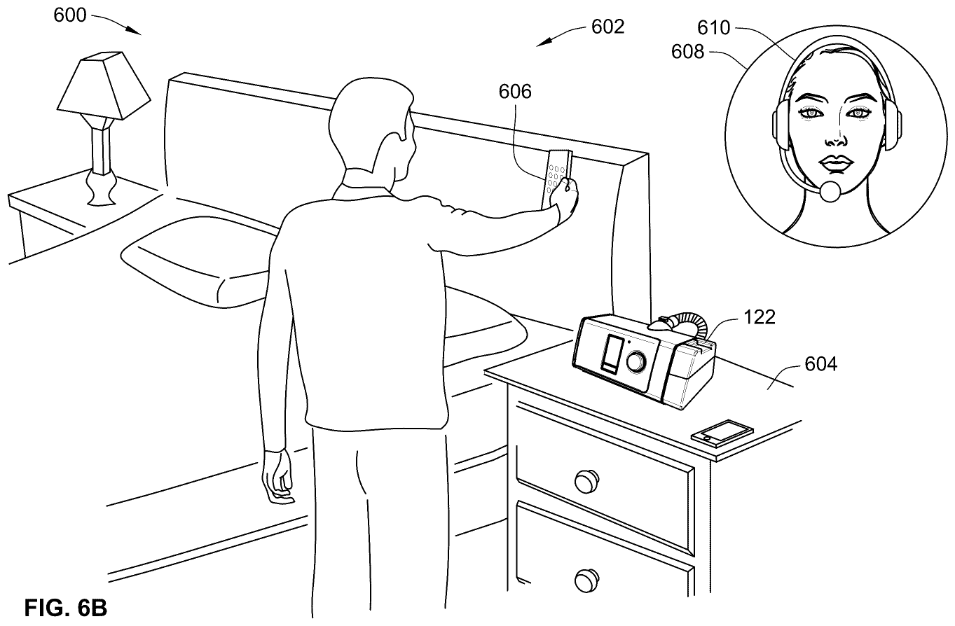

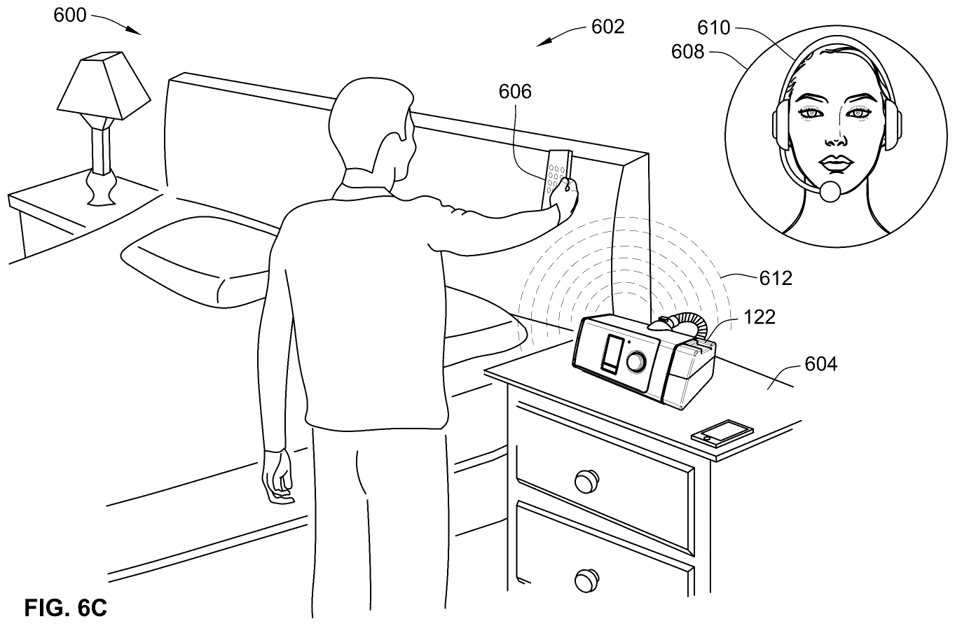

FIGS. 6A-6C illustrate an implementation where the respiratory therapy device 110 transmits the data for the compliance report 500 by generating an audible signal. In this implementation, compliance data is encoded into audio data that is reproducible as an audible signal via an audio transducer (e.g., a speaker, buzzer, etc.). The respiratory therapy device can operate the audio transducer to produce the audible signal once an audio connections has been established between an individual 600 and a third party 608. The third party 608 can detect the audible signal via the audio connection, and decode the audible signal to produce the compliance data that was encoded into the audio data.

As used herein, the term “compliance data” generally refers to any data that may be transmitted from the respiratory therapy system 100 (and/or a component of the respiratory therapy system 100, such as the respiratory therapy device 110) to a third party (such as a payor or another entity that prepares compliance reports for the payor) for purposes of quantifying and/or qualifying the individual's use of the respiratory therapy system 100, and/or the individual's adherence to a prescribed or desired regimen of use of the respiratory therapy system 100.

FIG. 6A illustrates a bedroom 602 of the individual 600, with the respiratory therapy device 110 sitting on a bedside table 604. The individual 600 is speaking to the third party 608 through the individual's smartphone 606. In the illustrated implementation, the third party 608 has their own telephone system 610, and an audio connection has been established between the smartphone 606 and the telephone system 610 of the third party 608. At this stage, the individual 600 and the third party 608 can talk to each other, no compliance data has been transmitted.

The audio connection between the individual and the third party 608 may be established in any suitable manner. For example, the third party 608 could place a call to the smartphone 606 of the individual 600, or the individual 600 could place a call to the telephone system 610 of the third party 608. In any example, once the audio connection between the smartphone 606 and the telephone system 610 has been established, the third party 608 can transmit instructions to the individual 600 and/or can receive the compliance data via the audible signal.

In FIG. 6B, the third party 608 has transmitted an instruction to the individual 600 to move the smartphone 606 within a specified distance of the respiratory therapy device 110, and to provide input to the respiratory therapy device 110 to initiate the process of transmitting the compliance data. The instruction is generally transmitted to the individual 600 via the audio connection but could also be transmitted to the individual 600 in other manners, such as via the respiratory therapy device 110 or any other suitable device.

As shown, the individual 600 has moved the smartphone 606 closer to the respiratory therapy device 110, and is providing input to the respiratory therapy device 110 to initiate the transmission. This input can be provided in any suitable fashion. For example, if the respiratory therapy device 110 has a physically operable control mechanism (e.g., a button, a switch, a knob, etc.), the individual 600 can operate the control mechanism to provide the input. In another example, if the respiratory therapy device 110 has a touch screen, the individual 600 can interact with the touchscreen to provide the input.

In FIG. 6C, the respiratory therapy device 110 is generating an audible signal 612 that can be transmitted to the third party 608 via the audio connection. The respiratory therapy device 110 will generally include an audio transducer, such as a speaker, a piezoelectric buzzer, a magnetic buzzer, etc. The respiratory therapy device 110 can operate the audio transducer to generate the audible signal 612. The audible signal 612 is detected by the smartphone 606 and transmitted to the telephone system 610 of the third party 608. The audible signal 612 can be decoded by the third party 608 to produce the compliance data.

Thus, the implementation illustrated in FIGS. 6A-6C allows for the compliance data transmitted to the third party 608 without the individual 600 having to connect their smartphone 606 to the respiratory therapy device 110, and without having to directly connect the respiratory therapy device 110 to the third party 608.

While the illustrated implementation shows the use of the smartphone 606, the individual 600 may generally use any suitable personal device to establish the audio connection with the third party 608, such as the smartphone 606, a tablet computer, a laptop computer, a desktop computer, a landline telephone, etc. Similarly, the third party 608 may use a device or system other than the telephone system 610, such as any suitable computing system and/or computing device. Moreover, the audio connection can be an audio-only connection, but could also be an audio-video connection. For example, the audio connection may be formed by the establishment of a video call between a computer of the individual 600, and a computer of the third party 608.

Further, the individual 600 involved in the transmission of the compliance data is not required to be the individual who actually uses the respiratory therapy device 110. For example, the individual 600 could be an aide that assists the individual using the respiratory therapy device 110. This aide could be a caretaker (e.g., a doctor, a nurse, a home health aide, etc.) a family member, or any other person that may assist the individual using the respiratory therapy device 110. The term “aide” is used herein to refer to any individual, other than the actual individual using the respiratory therapy device 110, that interacts with the respiratory therapy device 110 and/or assists with the transmission of the compliance data.

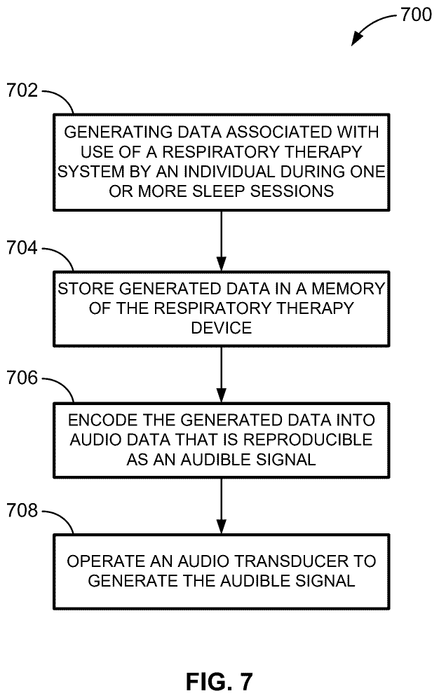

Referring now to FIG. 7, a flowchart of a method 700 for operating a respiratory therapy system (such as respiratory therapy system 100) is illustrated. Generally, a control system having one or more processors (such as control system 200 of system 10) is configured to carry out the steps of method 700. A memory device (such as memory device 204 of system 10) can be used to store machine-readable instructions that are executed by the control system to carry out the steps of method 700. The memory device can also store any type of data utilized in the steps of method 700. Generally, method 700 can be implemented using a system (such as system 10) that includes the respiratory therapy system, the control system, and the memory device, which may comprise the respiratory therapy device with the control system and memory device disposed therein.

Step 702 of method 700 includes generating data associated with use of the respiratory therapy system by an individual during one or more sleep sessions. The data that is generated can include data generated by the respiratory therapy system and/or sensors of the respiratory therapy system during use of the respiratory therapy system (such as flow rate values, therapy pressure values, time stamps associated with use, etc.), and can also include statistical data and other complementary data associated with use of the respiratory therapy system (such an average durations of use, amounts of respiratory events, amounts of air leak, indications of whether compliance thresholds have been met, identifiers of the respiratory therapy system and/or the respiratory therapy device, etc.).

In some implementations, the data generated in step 702 includes data generated only by processors of the control system of the respiratory therapy device. In other implementations, the data generated in step 702 includes data generated only by processors external to the respiratory therapy device (e.g., a computing device connected to the respiratory therapy device). In further implementations, the data generated in step 702 includes both data generated by processors of the control system of the respiratory therapy device, and data generated by processors external to the respiratory therapy device. In any of these implementations, step 704 of method 700 includes storing the generated data in the memory of the respiratory therapy device. The data will generally be stored as it is generated, such that steps 702 and 704 will at least partially be performed together.

Step 706 of method 700 includes encoding at least a portion of the generated data into audio data that is reproducible as an audible signal. In some cases, the audio data is stored in the memory of the respiratory therapy device. Any suitable digital modulation technique for encoding the portion of the generated data into the audio data can be used. In some implementations, keying techniques for encoding digital data into an analog signal can be used. These keying techniques can include frequency-shift keying (FSK, encoding digital data by modulating the frequency of the analog signal), phase-shift keying (PSK, encoding digital data by modulating the phase of the analog signal), and amplitude-shift keying (ASK, encoding digital data by modulating the amplitude of the analog signal). These keying techniques can also include techniques that modulate multiple characteristics of the analog signal (such as quadrature amplitude modulation, or QAM) and/or combinations of individual keying techniques. Other forms of digital modulation can also be used, such as orthogonal frequency-divisional multiplexing (OFDM), wavelet modulation, Trellis modulation (also known as trellis coded modulation or TCM), spread spectrum techniques, and others.

The portion of the generated data that is encoded into the audio data (e.g., the compliance data) can generally include any data that needs to be transmitted to a third party for compliance reporting purposes. In some implementations, the encoded data includes data associated with characteristics of the one or more sleep sessions, such as the average duration of use per sleep session, the average duration of use per day over a period of days that includes the one or more sleep session, the duration of use for each sleep session, the maximum therapy pressure for each sleep session and/or for the period of days, the minimum therapy pressure for each sleep session and/or for the period of days, the average therapy pressure for each sleep session and/or for the period of days, the total number of respiratory events experienced by the individual during each sleep session and/or during the period of days, the average number of respiratory events experienced by the individual per sleep session, an apnea-hypopnea index (AHI) for each sleep session and/or for the period of days, an apnea index (AI) for each sleep session and/or for the period of days, a hypopnea index (HI) for each sleep session and/or for the period of days, the total amount of air leak during each sleep session and/or during the period of days, the average amount of air leak per sleep session, and others.

In some implementations, the encoded data additionally or alternatively includes classifications of the value of any of the above characteristics and/or other characteristics. For example, instead of or in addition to a specific value of the average duration of use per sleep session (e.g., average duration of use equals 5.7 hours), the encoded data can include an indication of the number of sleep session where the duration of use was less than a threshold duration, and the number of sleep sessions where the duration of use was greater than the threshold duration (e.g., n sleep session with a duration of use less than 4 hours, and m sleep sessions with a duration of use greater than or equal to 4 hours). In another example, the encoded data can include an indication of whether the average duration of use per sleep session is “low,” “medium,” or “high.” Other categories/classifications can also be used.

In some implementations, the encoded data additionally or alternatively includes an indication of whether any of the above characteristics and/or other characteristics satisfy a threshold value. For example, instead of or in additional to a specific value of the average duration of use per sleep session or a classification of the average duration of use per sleep session, the encoded data can include an indication that the average duration of use per sleep session did satisfy a predetermined threshold (e.g., the user used the respiratory therapy system for at least a minimum amount of time as required by the third party), or an indication that the average duration of use per sleep session did not satisfy the predetermined threshold.

In some implementations, the encoded data additionally or alternatively includes an indication of whether a given sleep session satisfies a predetermined compliance threshold, and/or whether a certain period of time that includes one or more sleep session satisfies a predetermined compliance threshold. The compliance thresholds can be defined in any suitable manner. For example, a sleep session could be determined to satisfy the compliance threshold if the respiratory therapy system was operating for at least a minimum amount of time and the user interface was worn for at least a minimum amount of time. In another example, the period of time could be determined to satisfy the compliance threshold if the total duration of use during the period was at least a minimum amount of time.

In some implementations, the encoded data additionally or alternatively includes data that is associated with the compliance of the individual over a period of time that includes one or more sleep sessions. For example, the encoded data can include the number of days during the period of time, the number of sleep sessions during which the respiratory therapy device was used, the number of sleep sessions during which the respiratory therapy device was used for at least a threshold amount of time, the total number of hours of use of the respiratory therapy device during the period of time, the average duration of use of the respiratory therapy device per day during the period of time, the average duration of use of the respiratory therapy device per sleep session during the period of time, other characteristics or qualities of the period of time, or any combinations thereof.

In some implementations, the encoded data additionally or alternatively includes an identifier of the respiratory therapy device (and/or any other component of the respiratory therapy system). This identifier can be any suitable identifier, such as an ID number, an indication of the manufacturer, an indication of the model, etc.

In general, the encoded data can include any data that the third party needs for compliance purposes. The encoded data could include only the data needed by the third party, or the encoded data may include a larger amount of data that the third party analyzes to identify the data needed for compliance purposes.

Step 708 of method 700 includes operating an audio transducer to generate the audible signal. Any suitable audio transducer can be used, such as a speaker, a piezoelectric buzzer, a magnetic buzzer, etc. In some implementations, the audio transducer is located within the housing of the respiratory therapy device, but may additionally or alternatively be disposed or distributed in other locations.

The audible signal is decodable to produce the portion of the generated data that was encoded into the audio data. In some implementations, the audio signal is decodable by a service separate from the respiratory therapy device, which may be a personal device of the individual (e.g., the individual's smartphone), a personal device the aide of the individual (e.g., a smartphone of a caretaker, family member, partner, etc.), or a device of the third party that is connected to the personal device of the individual and/or aide by an audio connection. The audio connection may be an audio-only connection (e.g., a phone call over a cellular network, a phone call over a VoIP network, etc.) or an audio-video connection (e.g., a video call).

Thus, the third party to which the compliance data needs to be sent can connect to the individual (or the aide) over an audio connection (e.g., a phone call). The audio transducer is then operated to generate the audible signal, which is detectible by the third party via the audio connection. The third party can then decode the audible signal to produce the encoded compliance data.

The audible signal can have generally any form, so long as it can be decoded to produce the encoded data. For example, the audible signal could be a tone with a varying frequency, a varying amplitude, a varying phase, some other varying characteristic, or any combination thereof. In some implementations, the audio data includes markers separate from the compliance data, such that portions of the audible signal may be decoded to reveal these markers. The markers can be used for any suitable purposed, such as specifying the beginning of the transmission of the compliance data (e.g., audio that is decodable to state “Start” or “Begin”), specifying the end of the transmission of the compliance data (e.g., audio that is decodable to state “Stop” or “End”), identifying specific sleep session to which the encoded data refers (e.g., audio that is decodable to state “Sleep Session 1,” “Sleep Session of August 1^{st}”), etc.

In some implementations, the audible signal may include one or more human-perceivable words. In some of these implementations, instead of the markers discussed above, the audible signal can include the actual human-perceivable words, with no need to actually decode those portions of the audible signal. For example, the audible signal could include a specific word to identify the beginning of the audible signal (e.g., “Start” or “Begin”), and a specific word to identify the end of the audible signal (e.g., “Stop” or “End”). In another example, the audible signal could include specific words to identify specific sleep sessions to which the encoded data refers (e.g., “Sleep Session 1, “Sleep Session of August 1^{st}”, etc.).

In some implementations, background noise from the location of the respiratory therapy device (e.g., the individual's bedroom) can be detected and mitigated by the respiratory therapy device, by the personal device of the individual and/or the aide, by the third party device, etc. In one example, method 700 can further include analyzing the background noise using at least one audio sensor (e.g., a microphone of the respiratory therapy device, the individual's personal device, etc.). Method 700 can further include, based on the analysis, modifying the audio data and/or the generation of the audible signal. In some cases, the modifying includes modifying the audio data or the generation itself such that the volume of the audible signal as perceived by the third party device (or whichever device or entity is decoding the audible signal) is increased.

In some implementations, the encoding of the generated data and/or the operation of the audio transducer to generate the audible signal occur in response to the respiratory therapy system receiving some input. In general, this input will indicate that an audio connection has been established between (i) the personal device of the individual or the aide, and (ii) the third party. In some cases, the input is manual input provided by the individual and/or the aide, such as by interacting with a touchscreen or button of the respiratory therapy device. In other cases, the third party provides the input, such as by interacting with the respiratory therapy device via remote connection (separate from the audio connection). In further cases, the input could be input provided by the personal device that is prompted by action of the third party. For example, when the audio connection is established, the third party can transmit an audible signal over the audio connection, which is then reproduced by the personal device and detected by the respiratory therapy device. In additional cases, input from both the individual/aide and the third party may be required to initiate the process.

In some cases, the input can also be used to modify the audio data and/or the generation of the audible signal as needed. For example, the input can be indicative of the type of personal device that is being used by the individual or the aide, and the audio data/the audible signal can be adjusted as needed (such as by increasing the volume if it is determined the audible signal will not be as loud to the third party after being transmitted via the audio connection). In another example, the input can be indicative of whether the personal device is being operated in loudspeaker mode, which may require the volume of the audible signal to be increased or decreased.

In yet a further example, the input may be indicative of the type of audio connection that has been established between the personal device and the third party (e.g., an audio or video call over a wireless Internet connection, an audio or video call over a wired Internet connection, an audio or video call over a wireless cellular network, an audio call over a wired telephone network, etc.) The audio data and/or the generation of the audible signal can be adjusted as necessary (such as by increasing the volume of it is determined that the transmission of the audible signal over the audio connection may limit the volume as perceived by the third party).

In some cases, the respiratory therapy device could transmit instructions to the individual/aide and/or the third party after receiving the input. For example, the respiratory therapy device could transmit an instruction to the individual/aide to place the personal device within a certain distance of the respiratory therapy device. These instructions could be transmitted via the audio transducer, via a display of the respiratory therapy device, via the personal device, in other manners, or any combinations thereof.

In some implementations, receiving the input causes the respiratory therapy device to begin encoding the compliance data into the audio data. In these implementations, the data will only be stored after being generated. Receiving the input will cause the data to be into audio data and will cause the audio transducer to be operated to generate the audible signal. In other implementations, the data can be encoded as it is generated and/or received, and receiving the input causes the audio transducer to be operated to generate the audible signal.

In some implementations, the respiratory therapy device can identify the relevant data that is needed once the third party requires the data. For example, the respiratory therapy device can determine the last time an audible signal was generated for purposes of transmitting compliance data, and identify sleep sessions occurring after the generation of the last audible signal. Only data associated with those identified sleep session is encoded into the audio data to be reproduced as the audible signal. In some cases, the respiratory therapy device identifies the specific sleep session only after receiving an input to initiate the process.

The audio connection can be established by the individual/aide or the third party. For example, in some cases, the third party establishes the audio connection between the device of the third party (which can be considered a remote device from the perspective of the individual/aide) and the personal device. The audible signal can then be received by the third party, and decoded to produce the compliance data. The remote device of the third party could be a remote telephone system, a remote computer system, other types of systems, or any combinations thereof (e.g., a computer system connected to a telephone system that automatically decodes the audible signal to produce the compliance data). In some cases, the third party can transmit an instruction to the individual/aide to move the personal device within a specific distance from the respiratory therapy device, and can also transmit an instruction to operate the respiratory therapy device to generate the audible signal (e.g., an instruction to the individual/aide to press a button of the respiratory therapy device or interact with a touchscreen of the respiratory therapy device).

Generally, method 700 can be implemented using a system having a control system with one or more processors, and a memory device storing machine readable instructions. The control system can be coupled to the memory device, and method 700 can be implemented when the machine readable instructions are executed by at least one of the processors of the control system. Method 700 can also be implemented using a computer program product (such as a non-transitory computer readable medium) comprising instructions that when executed by a computer, cause the computer to carry out the steps of method 700.

One or more elements or aspects or steps, or any portion(s) thereof, from one or more of any of claims or Alternative Implementations below can be combined with one or more elements or aspects or steps, or any portion(s) thereof, from one or more of any of the other claims or Alternative Implementations or combinations thereof, to form one or more additional implementations and/or claims of the present disclosure.

### ALTERNATIVE IMPLEMENTATIONS

Alternative Implementation 1. A respiratory therapy device configured to supply pressurized air to an individual during one or more sleep sessions, the respiratory therapy device comprising: an audio transducer; a memory storing machine-readable instructions; and a control system including one or more processors configured to execute the machine-readable instructions to: generate data associated with use of the respiratory therapy device by the individual during the one or more sleep sessions; store the generated data in the memory; encode at least a portion of the generated data into audio data that is reproducible as an audible signal; and operate the audio transducer to generate the audible signal.

Alternative Implementation 2. The respiratory therapy device of Alternative Implementation 1, wherein the audible signal is decodable to produce the portion of the generated data.

Alternative Implementation 3. The respiratory therapy device of Alternative Implementation 2, wherein the audible signal is decodable by a device separate from the respiratory therapy device that is configured to detect the audible signal.

Alternative Implementation 4. The respiratory therapy device of Alternative Implementation 3, wherein the device separate from the respiratory therapy device is (i) a personal device of the individual, (ii) a personal device of an aide of the individual, (iii) a third party device connected to the personal device of the individual or of the aide via an audio connection, or (iv) any combination of (i)-(iii).

Alternative Implementation 5. The respiratory therapy device of Alternative Implementation 4, wherein the audio connection is an audio-only connection or a audio/video connection.

Alternative Implementation 6. The respiratory therapy device of any one of Alternative Implementations 1 to 5, wherein the transducer includes a speaker, a piezoelectric buzzer, a magnetic buzzer, or any combination thereof.

Alternative Implementation 7. The respiratory therapy device of any one of Alternative Implementations 1 to 6, wherein the portion of the generated data that is encoded into the audio data includes (i) an average duration of use per sleep session over the one or more sleep sessions, (ii) an average duration of use per day over a period of one or more days that includes the one or more sleep sessions, (iii) a duration of use for each respective sleep session of the one or more sleep sessions, (iv) a maximum pressure of the pressurized air during each respective sleep session, (v) a minimum pressure of the pressurized air during each respective sleep session, (vi) a total number of respiratory events experienced by the individual during each respective sleep session, (vii) an average number respiratory events experienced by the individual during each respective sleep session, (viii) an apnea-hypopnea index (AHI) value for each respective sleep session, (ix) an amount of air leak during each respective sleep session, (x) a classification of a value of any of (i)-(ix), (xi) an indication of whether a value of any of (i)-(ix) satisfies a respective threshold value, or (xii) any combination of (i)-(xi).

Alternative Implementation 8. The respiratory therapy device of any one of Alternative Implementations 1 to 7, wherein the portion of the generated data that is encoded into the audio data includes an indication of whether a compliance threshold was met for the one or more sleep sessions.

Alternative Implementation 9. The respiratory therapy device of any one of Alternative Implementations 1 to 8, wherein the portion of the generated data that is encoded into the audio data includes an indication of whether a compliance threshold was met for a period of time that includes the one or more sleep sessions.

Alternative Implementation 10. The respiratory therapy device of any one of Alternative Implementations 1 to 9, wherein the portion of the generated data that is encoded into the audio data includes an identifier of the respiratory therapy device.

Alternative Implementation 11. The respiratory therapy device of any one of Alternative Implementations 1 to 10, wherein the portion of the generated data that is encoded into the audio data is associated with a compliance of the individual over a period of time that includes the one or more sleep sessions.

Alternative Implementation 12. The respiratory therapy device of Alternative Implementation 11, wherein the portion of the generated data that is encoded into the audio data includes (i) a number of days during the period of time, (ii) a number of the one or more sleep sessions during which the respiratory therapy device was used, (iii) a number of the one or more sleep sessions during which the respiratory therapy device was used for at least a threshold amount of time, (iv) a total number of hours of use of the respiratory therapy device during the period of time, (v) an average duration of use of the respiratory therapy device per day during the period of time, (vi) an average duration of use of the respiratory therapy device per sleep session during the period of time, or (vii) any combination of (i)-(vi).

Alternative Implementation 13. The respiratory therapy device of any one of Alternative Implementations 1 to 12, wherein the portion of the generated data is encoded into the audio data using frequency-shift keying (FSK), phase-shift keying (PSK), amplitude-shift keying (ASK), or any combination thereof.

Alternative Implementation 14. The respiratory therapy device of any one of Alternative Implementations 1 to 13, wherein the one or more processors are further configured to execute the machine-readable instructions to: analyze, using at least one audio sensor, background noise of a location of the respiratory therapy device; and based on the analysis, modify (i) the audio data, (ii) the generation of the audible signal via the audio transducer, or (iii) both (i) and (ii).

Alternative Implementation 15. The respiratory therapy device of Alternative Implementation 14, wherein modifying the generation of the audible signal includes increasing a volume of the audible signal.

Alternative Implementation 16. The respiratory therapy device of any one of Alternative Implementations 1 to 15, wherein the audible signal includes (i) a tone with a varying frequency, (ii) a tone with a varying amplitude, (iii) a tone with a varying phase, or (iv) any combination of (i)-(iii).

Alternative Implementation 17. The respiratory therapy device of any one of Alternative Implementations 1 to 16, wherein the audible signal includes one or more human-perceivable words.

Alternative Implementation 18. The respiratory therapy device of any one of Alternative Implementations 1 to 17, wherein the one or more processors are configured to encode the portion of the generated into the audio data and operate the audio transducer in response to receiving an input.

Alternative Implementation 19. The respiratory therapy device of Alternative Implementation 18, wherein the input includes an indication that an audio connection has been established between (i) a personal device of the individual or of an aide of the individual, and (ii) a third party.

Alternative Implementation 20. The respiratory therapy device of Alternative Implementation 19, wherein the third party is a healthcare provider or a technician associated with the respiratory therapy device.

Alternative Implementation 21. The respiratory therapy device of Alternative Implementation 19 or Alternative Implementation 20, wherein the personal device is a smartphone, a tablet computer, a laptop computer, a desktop computer, or a landline telephone.

Alternative Implementation 22. The respiratory therapy device of any one of Alternative Implementations 19 to 21, wherein the one or more processors are further configured to execute the machine-readable instructions to transmit an instruction to place the personal device within a specific distance from the respiratory therapy device.

Alternative Implementation 23. The respiratory therapy device of Alternative Implementation 22, wherein the instruction is transmitted via the audio transducer, a display of the respiratory therapy device, the personal device, or any combination thereof.

Alternative Implementation 24. The respiratory therapy device of any one of Alternative Implementations 19 to 23, wherein the input includes an indication of an identity of the personal device, and wherein the one or more processors are further configured to execute the machine-readable instructions to, based on the identity of the personal device, modify (i) the audio data, (ii) the generation of the audible signal via the audio transducer, or (iii) both (i) and (ii).

Alternative Implementation 25. The respiratory therapy device of any one of Alternative Implementations 19 to 24, wherein the input includes an indication of whether the personal device is operating in a loudspeaker mode, and wherein the one or more processors are further configured to execute the machine-readable instructions to, based on the indication, modify (i) the audio data, (ii) the generation of the audible signal via the audio transducer, or (iii) both (i) and (ii).

Alternative Implementation 26. The respiratory therapy device of any one of Alternative Implementations 19 to 25, wherein the input includes an indication of a type of the audio connection between the personal device and the third party, and wherein the one or more processors are further configured to execute the machine-readable instructions to, based on the type of the audio connection, modify (i) the audio data, (ii) the generation of the audible signal via the audio transducer, or (iii) both (i) and (ii).

Alternative Implementation 27. The respiratory therapy device of Alternative Implementation 26, wherein the type of the audio connection is a wireless internet connection, a wired internet connection, a wired telephone connection, or a cellular network connection.

Alternative Implementation 28. The respiratory therapy device of any one of Alternative Implementations 19 to 27, wherein the input is a manual input that is received from the individual or from an aide of the individual.

Alternative Implementation 29. The respiratory therapy device of Alternative Implementation 28, wherein the manual input is received via an external button of the respiratory therapy device, a touch screen of the respiratory therapy device, or any combination thereof.

Alternative Implementation 30. The respiratory therapy device of any one of Alternative Implementations 19 to 29, wherein the input is received from the third party.

Alternative Implementation 31. The respiratory therapy device of Alternative Implementation 30, wherein the input is an audio signal generated by the personal device, and transmitted from the third party to the personal device via the audio connection.

Alternative Implementation 32. The respiratory therapy device of any one of Alternative Implementations 1 to 31, wherein the one or more processors are further configured to execute the machine-readable instructions to: determine when an immediately prior audible signal was generated; and identify at least one sleep session of the one or more sleep sessions occurring after the generation of the immediately prior audible signal, wherein the portion of the generated data that is encoded into the audio data includes only data from the identified at least one sleep session.

Alternative Implementation 33. The respiratory therapy device of any one of Alternative Implementations 1 to 32, wherein after encoding the portion of the generated data into the audio data, the one or more processors are further configured to execute the machine-readable instructions to store the audio data in the memory.

Alternative Implementation 34. The respiratory therapy device of any one of Alternative Implementations 1 to 33, further comprising: a housing defining an air inlet and an air outlet; and a blower motor disposed at least partially in the housing, the blower motor being configured to draw air into the housing through the air inlet and cause pressurized air to flow out of the housing through the air outlet.

Alternative Implementation 35. A method of transmitting compliance information using a respiratory therapy device configured to supply pressurized air to an individual during one or more sleep sessions, the method comprising: generating data associated with use of the respiratory therapy device by the individual during the one or more sleep sessions; storing the generated data in a memory of the respiratory therapy device; encoding at least a portion of the generated data into audio data that is reproducible as an audible signal; and operating an audio transducer of the respiratory therapy device to generate the audible signal.

Alternative Implementation 36. The method of Alternative Implementation 35, further comprising receiving an input that includes an indication that an audio connection has been established between (i) a personal device of the individual or of an aide of the individual, and (ii) a third party, and wherein the encoding of the portion of the generated data into the audio data and the operating of the audio transducer are performed in response to receiving the input.

Alternative Implementation 37. A method of communicating with a respiratory therapy device that is configured to supply pressurized air to an individual during one or more sleep sessions, the method comprising: establishing an audio connection between (i) a remote device and (ii) a personal device of the individual or of an aide of the individual; receiving an audible signal that is generated by the respiratory therapy device and transmitted to the remote device from the personal device; and decoding the audible signal to produce data associated with use of the respiratory therapy device by the individual during the one or more sleep session.

Alternative Implementation 38. The method of Alternative Implementation 37, wherein the remote device is a remote telephone system or a remote computer system.

Alternative Implementation 39. The method of Alternative Implementation 37, wherein the personal device and the respiratory therapy device are both located in an identical location during the generation of the audible signal by the respiratory therapy device.

Alternative Implementation 40. The method of Alternative Implementation 37, further comprising transmitting an instruction move the personal device within a specific distance from the respiratory therapy device.

Alternative Implementation 41. The method of Alternative Implementation 37, further comprising transmitting an instruction to operate the respiratory therapy device to generate the audible signal.

Alternative Implementation 42. A system comprising: a control system including one or more processors; and a memory having stored thereon machine readable instructions; wherein the control system is coupled to the memory, and the method of any one of Alternative Implementations 35-41 is implemented when the machine executable instructions in the memory are executed by at least one of the one or more processors of the control system.

Alternative Implementation 43. A system for personalized entrainment, the system including a control system configured to implement the method of any one of Alternative Implementations 35-41.

44. A computer program product comprising instructions which, when executed by a computer, cause the computer to carry out the method of any one of Alternative Implementations 35-41.

Alternative Implementation 45. The computer program product of Alternative Implementation 44, wherein the computer program product is a non-transitory computer readable medium.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations or alternative implementations according to aspects of the present disclosure may combine any number of features from any of the implementations described herein, such as, for example, in the alternative implementations described below.

What is claimed is:

1. A respiratory therapy device configured to supply pressurized air to an individual during one or more sleep sessions, the respiratory therapy device comprising:
an audio transducer;
a memory storing machine-readable instructions; and
a control system including one or more processors configured to execute the machine-readable instructions to:
generate data associated with use of the respiratory therapy device by the individual during the one or more sleep sessions;
store the generated data in the memory;
encode at least a portion of the generated data into audio data that is reproducible as an audible signal; and
operate the audio transducer to generate the audible signal.

2. The respiratory therapy device of claim 1, wherein:
(i) the audible signal is decodable to produce the portion of the generated data;
(ii) the transducer includes a speaker, a piezoelectric buzzer, a magnetic buzzer, or any combination thereof;
(iii) the portion of the generated data is encoded into the audio data using frequency-shift keying (FSK), phase-shift keying (PSK), amplitude-shift keying (ASK), or any combination thereof; or
(iv) any combination of (i)-(iii).

3. The respiratory therapy device of claim 2, wherein the audible signal is decodable by a device separate from the respiratory therapy device that is configured to detect the audible signal, the device separate from the respiratory therapy device being (i) a personal device of the individual, (ii) a personal device of an aide of the individual, (iii) a third party device connected to the personal device of the individual or of the aide via an audio connection, or (iv) any combination of (i)-(iii).

4. The respiratory therapy device of claim 1, wherein the portion of the generated data that is encoded into the audio data includes (i) an average duration of use per sleep session over the one or more sleep sessions, (ii) an average duration of use per day over a period of one or more days that includes the one or more sleep sessions, (iii) a duration of use for each respective sleep session of the one or more sleep sessions, (iv) a maximum pressure of the pressurized air during each respective sleep session, (v) a minimum pressure of the pressurized air during each respective sleep session, (vi) a total number of respiratory events experienced by the individual during each respective sleep session, (vii) an average number respiratory events experienced by the individual during each respective sleep session, (viii) an apnea-hypopnea index (AHI) value for each respective sleep session, (ix) an amount of air leak during each respective sleep session, (x) a classification of a value of any of (i)-(ix), (xi) an indication of whether a value of any of (i)-(ix) satisfies a respective threshold value, or (xii) any combination of (i)-(xi).

5. The respiratory therapy device of claim 1, wherein the portion of the generated data that is encoded into the audio data includes (i) an indication of whether a compliance threshold was met for the one or more sleep sessions, (ii) an indication of whether a compliance threshold was met for a period of time that includes the one or more sleep sessions, (iii) an identifier of the respiratory therapy device, or (iv) any combination of (i)-(iii).

6. The respiratory therapy device of claim 1, wherein the portion of the generated data that is encoded into the audio data is associated with a compliance of the individual over a period of time that includes the one or more sleep sessions and includes (i) a number of days during the period of time, (ii) a number of the one or more sleep sessions during which the respiratory therapy device was used, (iii) a number of the one or more sleep sessions during which the respiratory therapy device was used for at least a threshold amount of time, (iv) a total number of hours of use of the respiratory therapy device during the period of time, (v) an average duration of use of the respiratory therapy device per day during the period of time, (vi) an average duration of use of the respiratory therapy device per sleep session during the period of time, or (vii) any combination of (i)-(vi).

7. The respiratory therapy device of claim 1, wherein the one or more processors are further configured to execute the machine-readable instructions to:
analyze, using at least one audio sensor, background noise of a location of the respiratory therapy device; and
based on the analysis, modify (i) the audio data, (ii) the generation of the audible signal via the audio transducer, or (iii) both (i) and (ii).

8. The respiratory therapy device of claim 7, wherein modifying the generation of the audible signal includes increasing a volume of the audible signal.

9. The respiratory therapy device of claim 1, wherein the audible signal includes (i) a tone with a varying frequency, (ii) a tone with a varying amplitude, (iii) a tone with a varying phase, (iv) one or more human-perceivable words, or (v) any combination of (i)-(iv).

10. The respiratory therapy device of claim 1, wherein the one or more processors are configured to encode the portion of the generated into the audio data and operate the audio transducer in response to receiving an input that includes an indication that an audio connection has been established between (i) a personal device of the individual or of an aide of the individual, and (ii) a third party.

11. The respiratory therapy device of claim 10, wherein the third party is a healthcare provider or a technician associated with the respiratory therapy device, and wherein the personal device is a smartphone, a tablet computer, a laptop computer, a desktop computer, or a landline telephone.

12. The respiratory therapy device of claim 10, wherein the one or more processors are further configured to execute the machine-readable instructions to transmit an instruction to place the personal device within a specific distance from the respiratory therapy device, the instruction being transmitted via the audio transducer, via the personal device, via a display of the respiratory therapy device, or any combination thereof.

13. The respiratory therapy device of claim 10, wherein the input includes an indication of an identity of the personal device, and wherein the one or more processors are further configured to execute the machine-readable instructions to, based on the identity of the personal device, modify (i) the audio data, (ii) the generation of the audible signal via the audio transducer, or (iii) both (i) and (ii).

14. The respiratory therapy device of claim 10, wherein the input includes an indication of whether the personal device is operating in a loudspeaker mode, and wherein the one or more processors are further configured to execute the machine-readable instructions to, based on the indication, modify (i) the audio data, (ii) the generation of the audible signal via the audio transducer, or (iii) both (i) and (ii).

15. The respiratory therapy device of claim 10, wherein the input includes an indication of a type of the audio connection between the personal device and the third party, and wherein the one or more processors are further configured to execute the machine-readable instructions to, based on the type of the audio connection, modify (i) the audio data, (ii) the generation of the audible signal via the audio transducer, or (iii) both (i) and (ii).

16. The respiratory therapy device of claim 10, wherein the input is a manual input that is received from the individual or from an aide of the individual, and wherein the manual input is received via an external button of the respiratory therapy device, a touch screen of the respiratory therapy device, or any combination thereof.

17. The respiratory therapy device of claim 10, wherein the input is an audio signal generated by the personal device, and transmitted from the third party to the personal device via the audio connection.

18. The respiratory therapy device of claim 1, wherein the one or more processors are further configured to execute the machine-readable instructions to:
determine when an immediately prior audible signal was generated; and
identify at least one sleep session of the one or more sleep sessions occurring after the generation of the immediately prior audible signal,
wherein the portion of the generated data that is encoded into the audio data includes only data from the identified at least one sleep session.

19. The respiratory therapy device of claim 1, wherein after encoding the portion of the generated data into the audio data, the one or more processors are further configured to execute the machine-readable instructions to store the audio data in the memory.

20. The respiratory therapy device of claim 1, further comprising:
a housing defining an air inlet and an air outlet; and
a blower motor disposed at least partially in the housing, the blower motor being configured to draw air into the housing through the air inlet and cause pressurized air to flow out of the housing through the air outlet.
